(19) United States
(12) Patent Application Publication
(10) Pub. No.: US 2022/0081470 A1
(43) Pub. Date: Mar. 17, 2022

(54) THIAZOLYL PEPTIDES FOR THE TREATMENT NONTUBERCULOUS MYCOBACTERIAL INFECTIONS

(71) Applicants: FOUNDATION FOR NEGLECTED DISEASE RESEARCH, Bangalore (IN); NATIONAL CENTRE FOR POLAR AND OCEAN RESEARCH, Goa (IN)

(72) Inventors: Shridhar Narayanan, Bangalore (IN); Parvinder KAUR, Bangalore (IN); Naveen Kumar CN, Bangalore (IN); Rahul MOHAN, Goa (IN)

(21) Appl. No.: 17/420,524

(22) PCT Filed: Jan. 3, 2020
(86) PCT No.: PCT/IB2020/050021

§ 371 Date: Jul. 2, 2021

(30) Foreign Application Priority Data
Jan. 5, 2019 (IN) 201941000576

(51) Int. Cl. C07K 5/103; A61K 31/496; A61K 31/7036; A61K 31/7048; A61P 31/04

(52) U.S. Cl. CPC C07K 5/101; A61K 31/496; A61K 38/00; A61K 31/7048; A61P 31/04; A61K 31/7036

(57) ABSTRACT

The present invention provides novel thiazolyl peptide compounds and their pharmaceutically acceptable salts either alone or in combinations with Rifampicin, Amikacin and Clarithromycin against infections caused by Nontuberculous mycobacteria, especially Mycobacterium avium (M. avium) and Mycobacterium gordonae (M. gordonae).

### RELATED APPLICATION

This application is a National Stage of PCT/IB2020/050021, filed 3 Jan. 2020, titled THIAZOLYL PEPTIDES FOR THE TREATMENT NONTUBERCULOUS MYCOBACTERIAL INFECTIONS, published as International Patent Application Publication No. WO 2020/141478, which claims priority to and the benefit of the Indian provisional application 201941000576 filed 5 Jan. 2019, each of which are incorporated by reference herein in its entirety for all purposes.

### FIELD OF INVENTION

The present invention is related to novel thiazolyl peptide compounds and their pharmaceutically acceptable salts either alone or in combinations with Rifampicin, Amikacin and Clarithromycin against infections caused by Nontuberculous mycobacteria, especially Mycobacterium avium(M. avium) and Mycobacterium gordonae (M. gordonae).

### BACKGROUND OF THE INVENTION

Nontuberculous mycobacteria (NTM) are species other than those belonging to the Mycobacterium tuberculosis. NTM are generally free-living organisms that are ubiquitous in the environment. There have been more than 140 NTM species identified to-date. They can cause a wide range of infections, with pulmonary infections being the most frequent (65-90%). There is growing evidence that the incidence of NTM lung diseases and associated hospitalizations are on the rise, mainly in regions with a low prevalence of tuberculosis. A crucial clinical problem remains the evaluation of NTM significance in relation to the disease, especially in regard to the colonization of the respiratory tract in patients with residual lesions after tuberculosis or bronchiectasis. Clinical and radiographic pictures of mycobacteriosis, as well as therapy, have often similarities to those of tuberculosis. However, the treatment regimen should be individualized. In addition to antituberculosis drugs, other antibiotics are used more frequently [Advs Exp. Medicine, Biology—Neuroscience and Respiration (2017) 27: 19-25].

NTM are ubiquitous in the environment with the heaviest concentrations found in soil and water sources. They are associated with biofilm formation, thus resulting in resistance to disinfectants and antibiotics. Currently, there are more than 150 species of Mycobacterium and it is likely that more will be discovered. A full listing of recognized NTM can be found at www.bacterio.cict.fr/m/mycobacterium.html. By far, the most common organism associated with pulmonary disease is the Mycobacterium avium complex (MAC), a slow growing NTM that encompasses many subspecies including M. avium, M. silvaticum, M. hominissuis, and M. paratuberculosis, as well as the species M. intracellulare, M. arosiense, M. chimaera, M. colombiense, M. marseillense, M. timonense, M. bouchedurhonense, and M. ituriense. Mycobacterium kasassii, also a slow growing organism, is the second most common cause of pulmonary infections in the United States and is responsible for pockets of infection in England. Mycobacterium abscessus, is the most commonly isolated rapidly growing NTM and is the third most common cause of lung disease, but throws maximum treatment challenges. Although most NTM lung infections are caused by these three organisms, it is important to recognize that many other NTM may cause pulmonary disease in both immunocompetent and immunocompromised hosts [J Thorac Dis 2014; 6(3):210-220].

NTM infections can be serious or life threatening in vulnerable populations.

Nontuberculous mycobacteria (NTMs) have recently emerged as a new threat to human health. NTMs incidence has increased globally causing a wide range of illnesses, including TB-like pulmonary symptoms. NTMs are opportunistic pathogens and often cause difficult to treat infections, including multidrug resistant fatal infections requiring prolonged treatments. Most classical anti-TB drugs are ineffective on NTMs. Presently the NTM drug pipeline is remarkably low, calling for an urgent need to develop anti-NTM specific drugs. Available therapeutic options are poorly tolerated and present with adverse effects. US20170360816 provides a method for administering a liposomal complexed aminoglycoside comprising a lipid component of neutral lipids and aminoglycoside for delivery into the lungs. Administration of this composition involves aerosolizing of a mixture of free aminoglycoside and liposomal complexed aminoglycoside. This may be complex as it involves use of nebulizer to administer the therapeutic to the lung.

The present invention addresses methods of treating NTM infections in patients. The compound of Formula I is presented in Indian Patent 323089 (WO2011027290) and is incorporated herein by reference. The compound is elucidated to be useful for the treatment or prevention of multidrug resistant bacteria such as MRSA, VRE and Mycobacterium tuberculosis. In the instant invention, applicants provide the use of the compound in NTM infections which present different clinical symptoms and challenges.

### SUMMARY OF THE INVENTION

The present invention provides Formula I, a thiazolyl peptide as a potent inhibitor of nontuberculous mycobacteria, the said peptide given by the following structure:

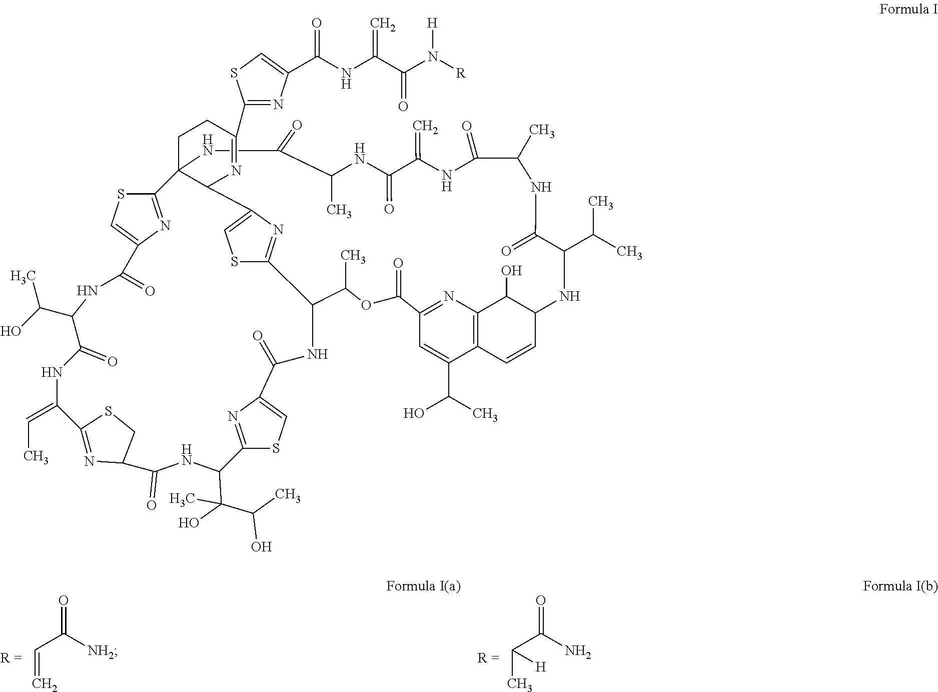

The composition of Formula I is disclosed in Indian Patent 323089 and is incorporated herein by reference in its entirety. Applicants have identified compound of Formula I (PM181108A—internal identifier) thiazolyl peptide as a potent inhibitor with an MIC of 1 μg/ml (for M. avium) and 2 μg/ml (for M. gordonae) and thus present it here as a therapeutic either alone or in combination for NTM infections. It was further characterized for its bactericidal activity in in-vitro combinations with Rifampicin, Amikacin and Clarithromycin. Thus, the disclosed thiazolyl peptide of Formula I is presented here as a therapeutic either alone or in combination with the additional compounds for Nontuberculous mycobacterial (NTM) infections.

### BRIEF DESCRIPTION OF DRAWINGS

FIGS. 1a and 1b. PM181108A exhibited Concentration X time dependent killing kinetics. (1a) M. gordonae. (1b) M. avium. PM181108A is a bactericidal compound, Emax ˜2.4 log 10 cfu/ml at 2 μg/ml, Cidality would further increase if tested at higher concentrations. Bactericidal definition is >2 log₁₀ cfu/ml kill.

FIGS. 2a, 2b, 2c, 2d, and 2e. PM181108A exhibited intracellular kill in the chronology of: (2a) M. nonchromogenicum (1.6 cfu log₁₀/ml)>(2b) M. kansasii (1.5)>(2c) M. avium (1.1)>(2d) M. gordonae (1.1)>(2e) M. intracellulare (0.6).

### BRIEF DESCRIPTION OF THE INVENTION

The present invention provides compounds of Formula I, the structure of which is provided below:

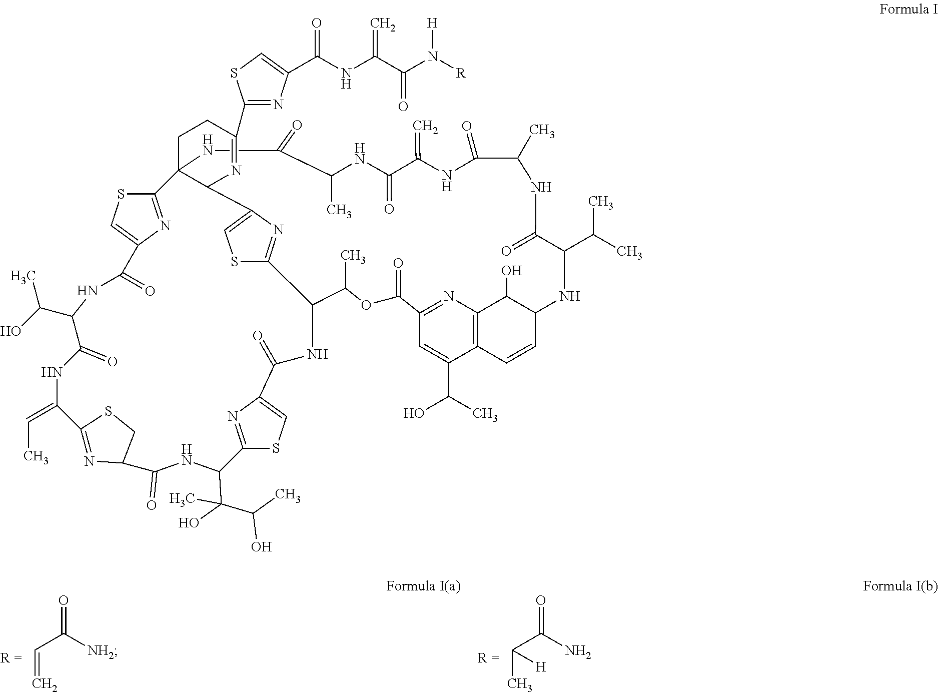

The present invention discloses compound Formula I (PM181108A—internal identifier) and is herein characterized for bactericidal activity and as combinations with Rifampicin, Amikacin and Clarithromycin against NTMs, especially against M. avium and M. gordonae species.

The compound of Formula I(a) is characterised by Molecular weight of 1649.5, molecular formula C₇₁H₈₃N₁₈O₁₈S₅ and that of Formula I(b) is characterised by Molecular weight of 1651.5, molecular formula C₇₁H₈₃N₁₈O₁₈S₅ and their ¹H NMR spectrum are provided in WO2011027290 which is incorporate herein by reference.

Compound of Formula I(a) and Formula I(b) are produced by cultivating microorganism species PM0626271/MTCC 5447 under submerged aerobic conditions in a nutrient medium containing carbon and nitrogen sources. in the fermented broth. The seed culture cultivation of PM0626271 is carried out at a temperature ranging from 25° C. to 36° C. and a pH of about 7.5 to 8 for 66 hours to 75 hours at 200 to 280 revolutions per minute.

In one embodiment the compound of Formula I designated as PM181108A has bactericidal activity against organisms that cause NTM infections. NTM strains causing NTM infections include Mycobacterium avium, Mycobacterium gordonae, Mycobacterium nonchromogenicum, Mycobacterium fortuitum, Mycobacterium abscessus, Mycobacterium intracellulare, Mycobacterium kansasii and Mycobacterium ulcerans which are covered in the instant invention.

In another embodiment, the compound of Formula I is administered alone to patients presenting clinical symptoms of NTM infections or proven presence of NTM.

In yet another embodiment, the compound of Formula I is administered in combination with Rifampicin, Amikacin or Clarithromycin to patients presenting clinical symptoms of NTM infections or proven presence of NTM. Rifampicin, Amikacin or Clarithromycin are administered simultaneously or sequentially with Compound of Formula I to patients presenting clinical symptoms of NTM infections or proven presence of NTM.

### Definitions

“Compounds of the invention” or “present invention” refers to the compounds of the present invention represented by general Formula (I) as herein defined, their derivatives, their analogs, their tautomeric forms, their stereoisomers, their bioisosters, their diastereomers, their polymorphs, their enantiomers, their appropriate N-oxides, their pharmaceutically acceptable salts, their pharmaceutically acceptable hydrates, their pharmaceutically acceptable solvates and pharmaceutically acceptable compositions containing them. The compounds of the present invention will be useful as microbicidal agents particularly in the treatment of NTM infections.

Uses

The compounds of the invention are useful for the treatment of infections in subjects, mammals in particular, including humans. In one embodiment, the compounds may be used for the treatment of infections of soft tissues, blood, skin, mouth, lungs, respiratory tract, urinary tract and reproductive tract.

In another embodiment, the compounds of the invention are useful for the treatment of infections caused by microorganisms, such as but not limited to bacterial infection, especially any Mycobacterium other than Mycobacterium tuberculosis. The present invention is used for the treatment of pulmonary disease caused by Mycobacterium abscessus, M. gordonae, M. fortuitum, M. non-chromogenicum, as well as Mycobacterium avium complex (MAC), a slow growing NTM that encompasses many subspecies including avium, silvaticum, hominissuis, and paratuberculosis, as well as the species M. intracellulare, M. arosiense, M. chimaera, M. colombiense, M. marseillense, M. timonense, M. bouchedurhonense and M. ituriense and M. kansassii.

Route of Administration

The compounds of the present invention are delivered to the subjects by forms suitable for each administration route. For example, the compounds are administered orally as tablets, capsules; parenterally as injections, inhaled as drops or as inhaler, topically as ointment, foams or administered as suppository. In a preferred embodiment, the route of administration is oral, parenteral, inhalation or topical. Topical or transdermal administration include powders, sprays, ointments, pastes creams, lotions, gels, solutions, patches and inhalants.

Dosage Forms

The composition of the present invention is presented in unit dosage form generally in an amount that produces a therapeutic effect in the subject.

The compounds of the present invention are administered at a daily dose that is the lowest dose effective to produce a therapeutic effect. Generally, the dosage is effective from about 0.0001 to about 100 mg per kg body weight per day. Preferably, the dosage will range from about 0.001 to 75 mg per kg body weight per day and more preferably, the dosage will range from about 0.1 to about 50 mg per kg body weight per day. Each unit dose may be, for example, 5, 10, 25, 50, 100, 125, 150, 200 or 250 mg of the compound of the invention. As per the requirement of the subject, the effective daily dose of the compound is administered as two, three, four or more sub-doses administered separately at appropriate intervals throughout the day, optionally in unit dosage forms.

Formulation

The antibacterial compositions of the present invention may be administered by any method known in the art. Some examples of suitable modes of administration include oral, intravenous, intramuscular topical or any other parenteral mode of administration.

In certain embodiments, the present invention is directed to a method of formulating compounds of the present invention in a pharmaceutically acceptable carrier or excipient and may be administered in a wide variety of different dosage forms e.g. tablets, capsules, sprays, creams, lotions, ointments, aqueous suspensions syrups, and the like. Such carriers may include one or more of solid diluents or fillers, sterile aqueous media, and various nontoxic organic solvents, etc.

For oral administration, tablets may contain various excipients such as one or more of microcrystalline cellulose, sodium citrate, calcium carbonate and the like, along with various disintegrants such as starch and certain complex silicates, together with granulation binders like polyvinylpyrrolidone, sucrose and the like. Solid compositions of a similar type may also be employed as fillers in gelatin capsules.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluents or solvent e.g. as solution in 1, 3 butane diol. Among the acceptable vehicles and solvents that may be employed are water, Ringer's solution and isotonic sodium chloride solution. In addition, sterile fixed oils are conventionally employed including synthetic mono or diglycerides. In addition, fatty acids such as oleic acid find in the preparation of injectables. These aqueous solutions may be suitable for intravenous injection purposes. The oily solutions may be suitable for intra articular, intramuscular, and/or subcutaneous injection purposes.

In another embodiment, the compounds of the present invention may be administered topically that include transdermal, buccal, or sublingual application. For topical applications, therapeutic compounds may be suitably admixed in a pharmacologically inert topical carrier such as a gel, an ointment, a lotion, and/or a cream. Such topical carriers may include water, glycerol, alcohol, propylene glycol, fatty alcohols, triglycerides, fatty acid esters, and/or mineral oils.

The timing of the administration of the pharmaceutical composition may also be regulated. For example the compounds may be administered intermittently or by controlled release.

The invention has been described with reference to various specific and preferred embodiments and techniques. However, it should be noted that many variations and modifications may be made while remaining within the scope of the invention. Likewise, the Examples provided herein are for illustrative purposes and should not be construed as limiting the invention in anyway.

Methods:

The compound of Formula I is isolated and purified from fermented broth of a microorganism belonging to Streptomyces species PM0626271/MTCC 5447. Isolation, purification, maintenance and fermentation of PM0626271 for the preparation of Formula I are carried out as per the protocols provided in WO2011027290 (Example 1 to Example 7) which is incorporated herein by reference.

PM181108A against Non-Tubercular Mycobacteria (NTM): List of the NTM strains used for the study: Mycobacterium avium, Mycobacterium gordonae, Mycobacterium nonchromogenicum, Mycobacterium fortuitum, Mycobacterium abscessus, Mycobacterium intracellulare, Mycobacterium kansasii and Mycobacterium ulcerans.

### Example 1.a) Determination of Minimum Inhibitory Concentration(MIC) in Non-Tuberculous Mycobacteria (NTM)

MICs against different species of NTM strains (Mycobacterium avium, Mycobacterium gordonae, Mycobacterium nonchromogenicum, Mycobacterium fortuitum, Mycobacterium abscessus, Mycobacterium intracellulare, Mycobacterium kansasii etc.) were determined by the standard broth dilution method according to CLSI document M24 [CLSI]. Briefly, the test compounds were dissolved in DMSO, serially double-diluted in a 10-concentration dose response (10-DR) ranging from 256-0.5 g/mL in 96-well plates. Middlebrook 7H9 broth (supplemented with 10% ADC) complete media was used for the assay. Mtb culture was added as 200 μl in each well to all columns except the media control (200 μl of media was added) column to give a final inoculum of 3-7×10⁵ cfu/ml. The QC included: media controls, growth controls, and the reference drug inhibitors (Rifampicin and Isoniazid). The assay plates were incubated at 37° C., resazurin dye was added on 6th day, and the results were noted on the 7^{th} day as colorimetric readout. The blue wells indicated inhibition of growth, while the pink wells indicated uninhibited growth. MIC was the minimum concentration of molecules that completely inhibited the colorimetric growth of bacteria. MIC assays were carried out three times in duplicate.

Results and Conclusion: The MIC values of PM181108A against NTM strains was in the range of 0.06 to 4 μg/ml (Tables 1a and 1b).

b) Minimum Bactericidal Concentration (MBC):

MBC was determined against NTM strains by serial 10-fold dilution of these tubes using phosphate buffer saline (0.1 M, pH 7.4) as a diluent. Each dilution (0.5 mL) was plated in triplicate onto Middlebrook 7H10 agar supplemented with 10% OADC and incubated at 37° C. The plates were counted for CFU on day 4 to day 21 of incubation for different strains, as per the fast or the slow growing NTMs. MBC was taken as the lowest concentration that killed 99.9% of the initial M. tuberculosis inoculum.

TABLE 1a
PM181108A activity against different NTM strains: MIC, MBC, and intracellular
efficacy.

| NTM | M.abscessess | | | M.avium | | | M.fortuitum | | |
|---|---|---|---|---|---|---|---|---|---|
| Compounds | MIC | MBC | IC Eₘₐₓ | MIC | MBC | IC Eₘₐₓ | MIC | MBC | IC Eₘₐₓ |
| PM181108A | 1 | 16 | 0.00 | 1 | 1 | 1.09 | 4 | >16 | 0.00 |
| Thiostrepton | 0.5 | 32 | 0.00 | 1 | 2 | 1.19 | 2 | >32 | 0.00 |
| Moxifloxacin | 2 | 4 | 1.00 | 4 | 8 | 1.00 | <0.06 | 0.25 | 1.60 |
| Rifampicin | 16 | 32 | 0.00 | 0.25 | 1 | 1.04 | 4 | >32 | 0.00 |
| Clarithromycin | 2 | 32 | 0.70 | 0.5 | 2 | 1.00 | 32 | >32 | 0.35 |
| Amikacin | 4 | 16 | ND | 2 | 8 | ND | 2 | 2 | ND |

| | | | |
|---|---|---|---|
| Standard of | Clarithromycin | All anti TB | Moxifloxacin/FQ |
| Care (SoC) | Azithromycin | | Rifabutin |
| | Bedaquiline (BDQ) | | |

TABLE 1b
PM181108A activity against different NTM strains: MIC, MBC, and intracellular
efficacy.

| NTM | M.gordonae | | | M.intracellulare | | | M.kansasii | | | M.nonchromogenicum | | |
|---|---|---|---|---|---|---|---|---|---|---|---|---|
| Compounds | MIC | MBC | IC Eₘₐₓ | MIC | MBC | IC Eₘₐₓ | MIC | MBC | IC Eₘₐₓ | MIC | MBC | IC Eₘₐₓ |
| PM181108A | 0.5 | 2 | 1.08 | 0.06 | 1 | 0.62 | 1 | 4 | 1.50 | 0.5 | 2 | 1.58 |
| Thiostrepton | 0.5 | 2 | 0.72 | 0.13 | 1 | 0.67 | 1 | 16 | 1.56 | 0.5 | 16 | 0.49 |
| Moxifloxacin | 2 | 4 | 1.23 | 0.5 | 1 | 1.46 | 2 | 8 | 1.93 | 2 | 16 | 0.34 |
| Rifampicin | 0.25 | 8 | 1.36 | 0.06 | 0.25 | 0.44 | 0.13 | 16 | 1.33 | 0.13 | 32 | 0.82 |
| Clarithromycin | 0.5 | 1 | 1.26 | 0.13 | 0.5 | 1.78 | 0.5 | 2 | 0.91 | 0.25 | 4 | 0.82 |
| Amikacin | 2 | 8 | ND | 1 | 2 | ND | 4 | 16 | ND | 2 | 8 | ND |

| | | | | |
|---|---|---|---|---|
| Standard of | Clarithromycin | Clarithromycin | Clarithromycin | All anti TB |
| Care (SoC) | Rifampicin | Rifampicin | Rifampicin, | |
| | | | Isoniazid | |
| | Ethambutol | Ethambutol | Ethambutol | |

Results and Conclusion: The MBC values of PM181108A against NTM strains was in the range of 1 to 4 μg/ml, with 2 to 4 fold of MIC (Tables 1a and 1b); except for the M. fortuitum NTM strain (>16 μg/ml).

### Example 2. Drug Combination Studies with the Current SOC

In vitro drug interaction study for PM181108A was performed as described previously (J Med Chem 56(23):9701-8, 2013). Briefly, the synergistic/additive/antagonist interactions of test molecule were tested with currently known anti-NTM drugs against NTM strains (RIF, AMK, CLAR, and MOX), by determining the MICs of the test molecule and anti-TB drugs alone/combination in 96-well plates by checkerboard method. Each combination was prepared such that the middle concentration of each molecule equalled its MIC. Serial dilutions were made in subsequent wells. The respective NTM cultures were added as 200 μl at an inoculum of approximately 3-8×10⁵ CFU/ml in each well. The plates were incubated at 37° C./6 days. Resazurin was added on 6^{th} day and continued incubation, the results were read by colorimetric inspection. MICs of each drug alone and in combination were described where the lowest concentrations showing no visible colour change from blue to pink (i.e. no growth of NTMs) were considered as the minimum inhibitory concentrations (MIC). The combinatorial reductions in MICs were used to calculate the fractional inhibitory concentration (FIC). Fractional inhibitory concentration indices (FIC) were interrupted as follows: ≤0.5, synergism; >0.5-4.0, addition or indifference; and >4.0, antagonism. PM181108A was found to show no antagonism with any of the SOC. None of the combinations had FIC >4.0.

Results and Conclusion: The compound demonstrated synergy or additive effect with the SoC. None of the SoC combination showed any kind of antagonism with PM181108A (Table 2). PM181108A was found to be either synergistic or additive in combination with any of the SOC.

TABLE 2
Invitro combination studies with Rifampicin, Amikacin and Clarithromycin
Invitro combination studies with Rifampicin, Amikacin and Clarithromycin

| | | M. avium | | | M. gordonae | | |
|---|---|---|---|---|---|---|---|
| | | Individual | | | Individual | | |
| Sl.no | Compound | MIC(ug/ml) | FIC index | Outcome | MIC(ug/ml) | FIC index | Outcome |
| | PM181108A | 1 | | | 2 | | |
| 1 | Rifampicin | 0.5 | 0.53 | Additive | 0.5 | 0.45 | Synergy |
| 2 | Amikacin | 4 | 0.80 | Additive | 4 | 0.90 | Additive |
| 3 | Clarithromycin | 1 | 0.46 | Synergy | 1 | 0.46 | Synergy |

### Example 3. Mycobactericidal Killing Kinetics Activity of PM181108A on Replicating NTMs

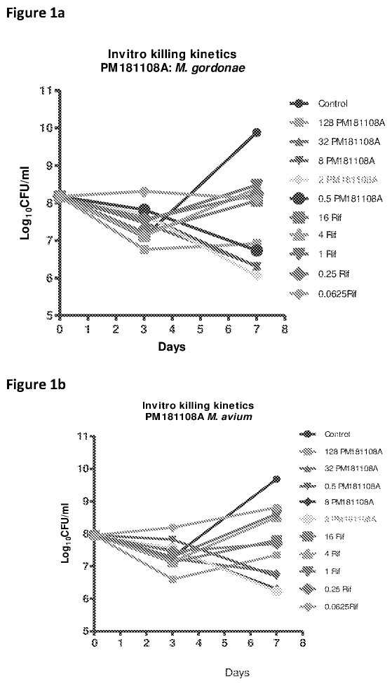

Killing kinetics assay on replicating population of NTMs was performed as described earlier (Antimicrobial Agents and Chemotherapy. 47: 2118-2124.2003). The respective NTM culture was inoculated at ˜3-8×10⁷ cfu/mL inoculum in fresh Middlebrook 7H9 complete medium containing varying concentrations of the compound PM181108A (0.015-32 ug/mL). The cultures were incubated at 37° C. for different time points, and enumerated respectively. For CFU enumeration, aliquots from the cultures containing different concentrations of the compounds were collected at day-3, day-7 and day-14 and plated at various dilutions (10⁻¹ to 10⁻⁸) to get countable colonies. Rifampicin was used as the quality control for the assay. The data was analysed and plotted as log₁₀ cfu/mL at day-3, day-7 and day-14 at as a function of concentration of PM181108A to calculate the range of concentration that shows killing potential. PM181108A, a bactericidal compound, showed an Emax=2.4 log₁₀ cfu/ml at 32 mg/ml (FIG. 1).

### Example 4. Cytotoxicity of PM181108A on THP-1 and HepG2

Cytotoxicity of the compound was tested on PMA-activated HepG2 and THP-1 macrophage cell lines (Antimicrobial Agents and Chemotherapy. 47: 2118-2124.2003)). THP-1 monocytes (ATCC TIB-202) were maintained in the RPMI 1640 medium supplemented with 2 mM 1-glutamine and 10% heat-inactivated foetal bovine serum (FBS) at 37° C. in a humidified atmosphere of 5% CO₂. FBS was obtained from Life Technologies. Resazurin, and trypan blue were purchased from Sigma-Aldrich.

THP-1 cells in RPMI were activated using 50 nM of phorbol 12-myristate 13-acetate for 48-72 hours at 37° C./5% CO₂. Post maturation of THP-1 cells into Macrophages, cells were exposed to test compound PM181108A was added at 2-fold concentrations (64-0.025 ug/ml) on the respective cell lines at 37° C./5% CO₂ for 48 hrs. Post incubation, resazurin dye was added at 25 mg/ml concentration with equal volume of RPMI media and further incubated for 24 hours. The colorimetric readings were taken after addition of resazurin dye.

Results and conclusion: PM181108A did not demonstrate any toxicity to THP-1 cells even up to 128×MIC (cytotoxicity >64 μg/ml) the maximum concentration tested.

### Example 5. Intracellular Efficacy of PM181108A Against NTMs in THP-1 Macrophages

To test drug efficacy against slow or non-replicating bacilli in the intracellular compartment, THP-1 cells were grown in RPMI medium supplemented with 100 mM sodium pyruvate, 200 mM L-glutamine, 3.7 g of sodium bicarbonate (Sigma) per litre, and 10% foetal calf serum (Gibco-BRL Life Technologies) without any antibiotics. The macrophages were seeded in 96-well plates at a density of approximately 5×10⁵ cells/flask, incubated overnight, and were induced by 50 nM phorbol 12-myristate 13-acetate (PMA) to achieve macrophage differentiated phenotypesat 37° C./48-72 hr/5% CO₂ atmosphere. After 48 hr of activation, the THP-1 macrophages were infected with respective NTM strains at a multiplicity of infection (MOI) of 1:10/2 h at 37° C. with 5% CO₂. The macrophage monolayers were washed twice with 3 ml of phosphate-buffered saline (+Ca²⁺+Mg²⁺) to remove the free bacteria. Sets of triplicate wells were lysed (0.05% SDS) at specific timepoints, and enumerated to estimate the numbers of intracellular NTM 2 h post-infection.

The remaining wells of the assay plate were used for testing dose response of PM181108A (64-4-1 μg/ml), drug control rifampicin (at 16-4-1 μg/ml) as well as the infection controls in triplicate wells at respective concentrations. The residual intracellular viable mycobacteria were enumerated at 0, 3, 5, and 7 day on Middlebrook 7H11 agar plates. The intracellular mycobacterial killing rates of rifampicin were determined by nonlinear regression analysis (95% confidence limits). Inhibitory sigmoidal curves were generated by plotting the log₁₀ cfu/ml against the C_{broth}/MIC ratio and the AUC/MIC ratio. PM181108A demonstrated on Day-7 (FIG.-2) a 1.6 log₁₀ drop at 32 μg/ml concentration.

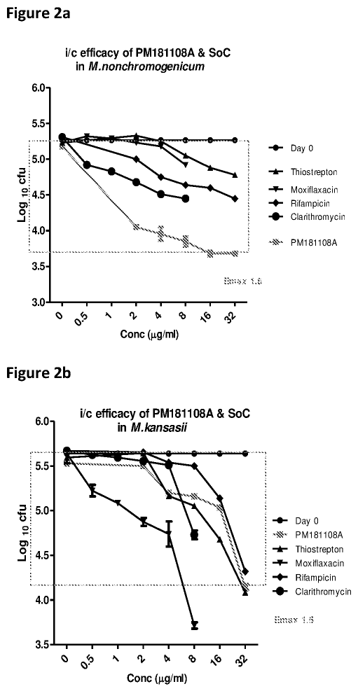

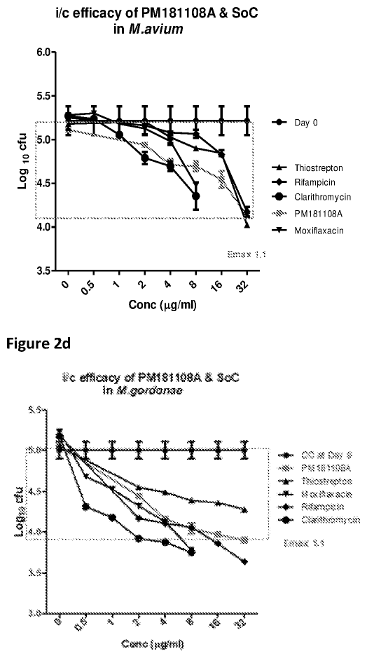

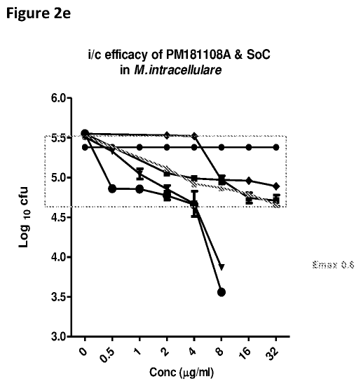

Results: PM181108A was equally potent or better than the SoC against different NTMs, in the order of: M. nonchromogenicum(1.58)>M. kansasii(1.5)>M. avium (1.09)>M. gordonae(1.08)>M. intracellulare(0.62)(FIG. 2). M. abscessus is also effective than the SoC (data not provided).

### Example 6. Resistance Mutants of PM181108A Against NTMs

Two representative NTMs M. gordonae and M. avium: 7H9 agar was prepared and autoclaved. Drug containing 7H9 agar (10% ADC) plates were made by addition of the compound to molten agar once it reached ˜45° C. 7H9 agar containing 100× and 300× concentration of compound was prepared (MIC being 0.5 μg/ml) and incubated. Both the cultures were grown in 7H9 containing ADC at 37° C., 100 rpm for 2-3 days. The cell number being approximately ˜10⁹ cfu/ml, 0.5 ml of culture is plated on each drug containing plates (2 drug containing plates of each concentration).

Results and conclusion: Both M. gordonae and M. avium did not yield any resistance mutants against PM181108A.

1. A composition comprising a thiazolyl peptide of Formula I(a) or I(b): 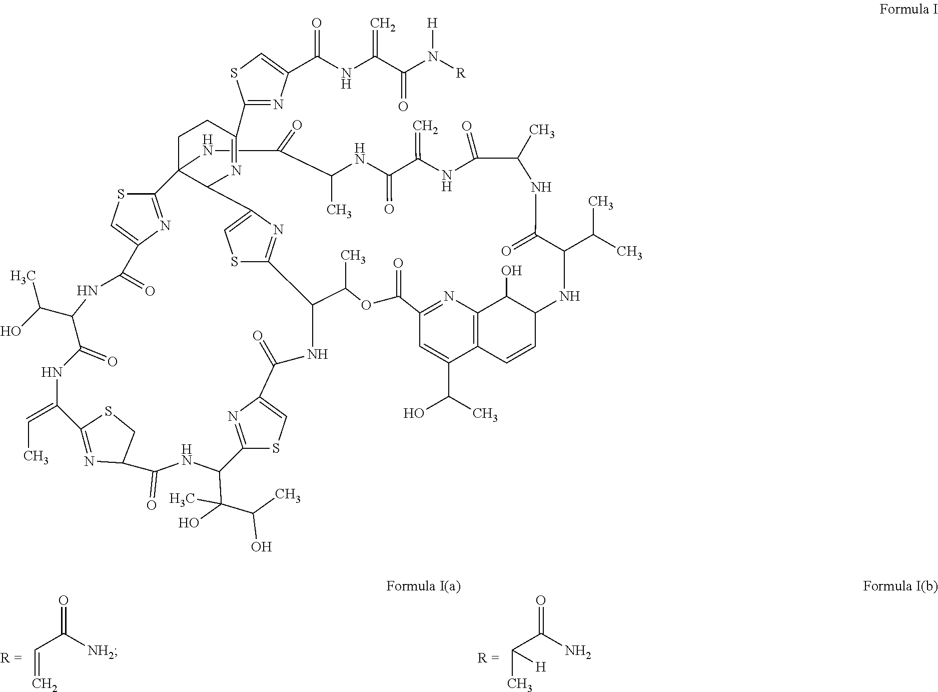
and an active ingredient selected from the group consisting of Rifampicin, Amikacin and Clarithromycin.

2. A pharmaceutical composition comprising an effective amount of a thiazolyl peptide of Formula I(a) or Formula I(b) 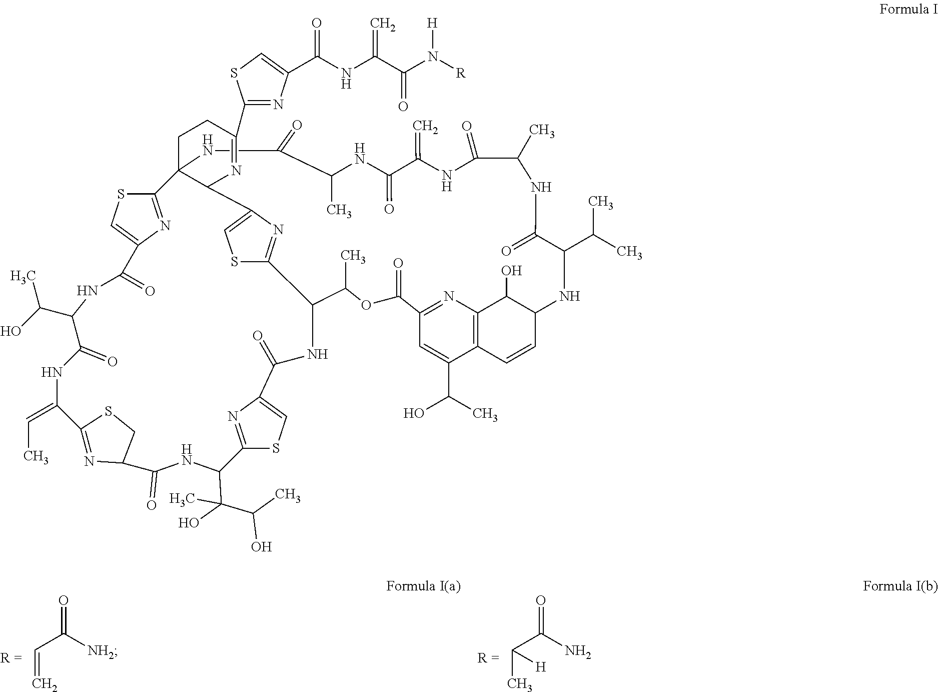
at least one pharmaceutically acceptable excipient, and an active ingredient selected from the group consisting of Rifampicin, Amikacin and Clarithromycin.

3. (canceled)

4. The pharmaceutical composition of claim 2 wherein the composition is formulated for a route of administration selected from the group consisting of oral, parenteral, inhalation, and topical.

5. (canceled)

6. A method of preventing or treating a non-tuberculous mycobacterial (NTM) infection in a subject, the method comprising administering to a subject in need thereof a composition comprising a thiazolyl peptide of Formula I(a) or I(b):

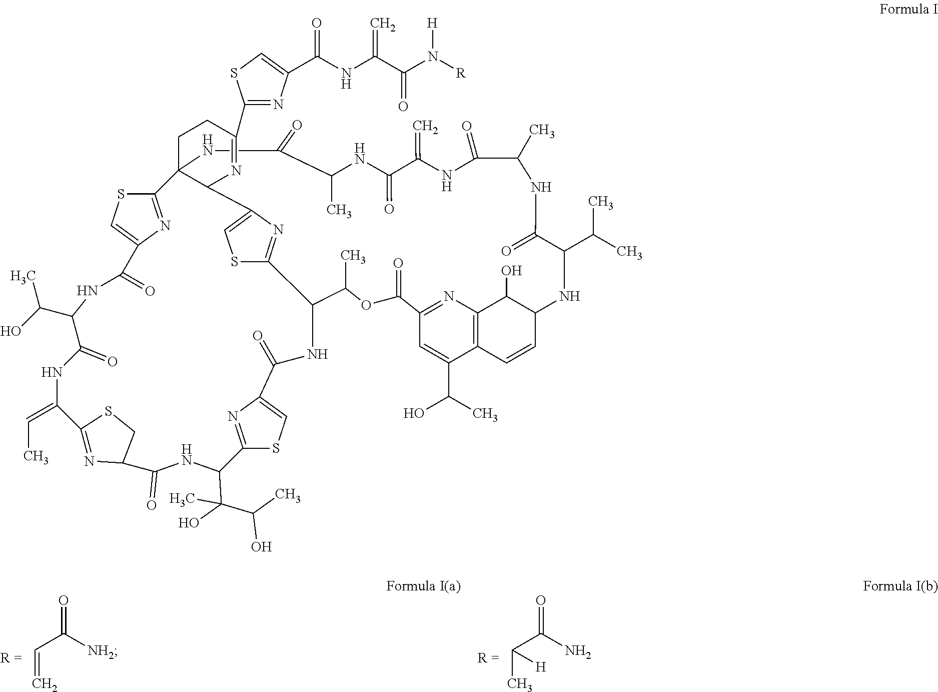

7. (canceled)

8. The method of claim 6, wherein the route of administration of the composition is selected from the group consisting of oral, parenteral, inhalation, and topical.

9. The method of claim 6, wherein the NTM infection is caused by Mycobacterium species selected from the group consisting of M. avium, M. silvaticum, M. hominissuis, M. paratuberculosis, M. intracellulare, M. arosiense, M. chimaera, M. colombiense, M. marseillense, M. timonense, M. bouchedurhonense, and M. ituriense.

11. The method of claim 6, further comprising administering an active ingredient selected from the group consisting of Rifampicin, Amikacin and Clarithromycin.

12. The method of claim 11, wherein the active ingredient is administered simultaneously.

13. The method of claim 11, wherein the active ingredient is administered sequentially.

14. The method of claim 11, wherein the route of administration of the composition is selected from the group consisting of oral, parenteral, inhalation, and topical.

15. The method of claim 11, wherein the NTM infection is caused by a Mycobacterium species selected from the group consisting of M. avium, M. silvaticum, M. hominissuis, M. paratuberculosis, M. intracellulare, M. arosiense, M. chimaera, M. colombiense, M. marseillense, M. timonense, M. bouchedurhonense, and M. ituriense.15.

16. The method of claim 6, wherein the composition further comprises an active ingredient selected from the group consisting of Rifampicin, Amikacin and Clarithromycin.

14. The method of claim 16, wherein the route of administration of the composition is selected from the group consisting of oral, parenteral, inhalation, and topical.

15. The method of claim 16, wherein the NTM infection is caused by a Mycobacterium species selected from the group consisting of M. avium, M. silvaticum, M. hominissuis, M. paratuberculosis, M. intracellulare, M. arosiense, M. chimaera, M. colombiense, M. marseillense, M. timonense, M. bouchedurhonense, and M. ituriense.
